(19) United States
(12) Patent Application Publication
(10) Pub. No.: US 2024/0262864 A1
(43) Pub. Date: Aug. 8, 2024

(54) ECHINOCANDIN DRUG IMPURITY, AND PREPARATION AND PURIFICATION METHODS AND USE THEREOF

(71) Applicant: Shanghai Techwell Biopharmaceutical Co., Ltd, Shanghai (CN)

(72) Inventors: Rongguo SHI, Shanghai (CN); Min CHEN, Shanghai (CN); Yushan GUO, Shanghai (CN); Yan LI, Shanghai (CN); Xiaoming JI, Shanghai (CN)

(21) Appl. No.: 18/006,110

(22) PCT Filed: Jun. 17, 2022
(86) PCT No.: PCT/CN2022/099564

§ 371 Date: Jan. 19, 2023

(30) Foreign Application Priority Data
Sep. 9, 2021 (CN) 202111057760.2

(51) Int. Cl. C07K 7/56; C07K 1/16

(52) U.S. Cl. CPC C07K 7/56; C07K 1/16

(57) ABSTRACT

The present disclosure provides an echinocandin drug impurity and preparation and purification methods and use thereof. The echinocandin drug impurity has a structure shown in Formula I:

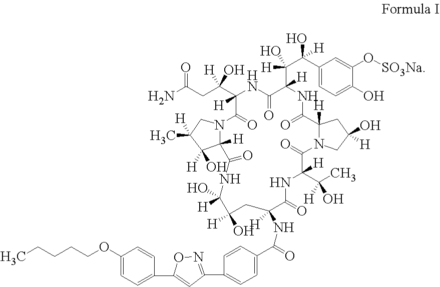

In the present disclosure, micafungin sodium is reacted with a protonic acid aqueous solution to obtain an echinocandin drug impurity, and a high-purity echinocandin drug impurity is obtained after chromatographic purification. The present disclosure overcomes problems that an echinocandin drug impurity is prone to being degraded during the purification, and purification difficulty is high, and an HPLC purity of the obtained echinocandin drug impurity may reach 90% or more. The echinocandin drug impurity may be used as a reference substance for establishing an analysis method for use in echinocandin drug quality control.

### TECHNICAL FIELD

The present disclosure relates to the technical field of medicine, and provides an echinocandin drug impurity and preparation and purification methods and use thereof.

### BACKGROUND

Micafungin sodium is a new-generation echinocandin antifungal drug, is mainly used to treat fungemia, respiratory tract mycoses and gastrointestinal tract mycoses caused by aspergilli and candida, and also used to prevent hematopoietic stem cell patients from being infected with the aspergilli and candida, is a type of echinocandins antifungals and can inhibit synthesis of filamentous fungi and yeast β(1,3)-glucan.

In recent years, fungal infection incidence rate and fatality rate are also on the increase, especially, critical patients are under a deadly threat. Micafungin sodium, a type of echinocandin drugs, has a good antifungal activity and is a priority of drugs for treating infections caused by candida and aspergilli, which has a good treatment effect, smaller influence on human cells and has clinical effects of low toxicity and high effectiveness.

An echinocandin drug impurity (shown in Formula I) is a by-product in a degradation process of micafungin. A deep research is conducted on the impurity and discovers that the impurity is similar to a structure of the micafungin sodium, has a plurality of chiral centers and a plurality of functional groups such as peptide bonds and is extremely unstable to acid, alkali, heat, light. There is no literature report related to a method for preparing the impurity at present.

In a research and development process of drugs, analysis for impurities is the key. Impurities are closely related to drug quality, security and stability. A pathway by which impurities are produced may be understood through preparation and structure confirmation of the impurities, and a basis is provided for a drug synthesis process route and improvement on production processes. The impurities may be applied to drug quality control and are used as reference substances for establishing an analysis method. As for a pharmaceutical producing enterprise, the impurities need to be strictly controlled. A related research on the echinocandin drug impurity may be used for qualitative and quantitative analysis of an impurity during production of micafungin sodium and thus provides technical support for a subsequent quality research on the micafungin sodium to improve a quality standard of the micafungin sodium.

Therefore, it is urgent now to provide a high-efficiency and convenient method for preparing an echinocandin drug impurity (shown in Formula I).

### SUMMARY

The inventor conducts a deep research on a process for preparing an echinocandin drug impurity. Referring to literature of Journal of Synthetic Organic Chemistry 2006, 64, 12] and a patent U.S. Pat. Nos. 6,107,458, 7,199,248, during preparation of micafungin sodium, it is discovered that there is an unknown impurity at a position RRT1.1 in an HPLC graph, a peak area ratio of the impurity to the micafungin sodium is 0.2%. In a subsequent recrystallization stage, the impurity cannot be removed effectively, and a certain proportion of the unknown compound still remains. In an experimental process, it is unexpectedly discovered that when a micafungin sodium sample is put in an acidic aqueous solution, a content of the unknown compound impurity tends to increase. Thus, the present disclosure is conceived, and a synthetic experiment is designed, that is, the micafungin sodium is made to reach with acid to prepare the impurity.

The inventor uses the micafungin sodium as a raw material and reacts it with an aqueous hydrochloric acid solution in a room temperature, and a purity of the obtained unknown impurity RRT1.1 is low, which is merely 3%. The inventor optimizes reaction conditions through a large number of experiments and finally determines an optimal process for synthesizing the unknown impurity RRT1.1. It is discovered that when the impurity is purified by using a chromatographic column, the impurity is prone to being degraded during the purification, so purification difficulty is quite high. By optimizing purification conditions and determining an optimal purification process, the unknown impurity RRT1.1 with an HPLC purity reaching 90% or more is obtained. Through structural identification for the unknown impurity, it is determined that the impurity is a compound represented by Formula I. It is discovered that a structural difference between the impurity and the micafungin sodium is that a hydroxyl group at a position C-21 of the impurity is an S configuration, but a hydroxyl group at a position C-21 of the micafungin sodium is an R configuration. In the preparation process, the impurity can be generated under an acidic condition, and through analysis, a generation path of the impurity is a product of racemization of the hydroxyl group at a position C-21 under the acidic condition.

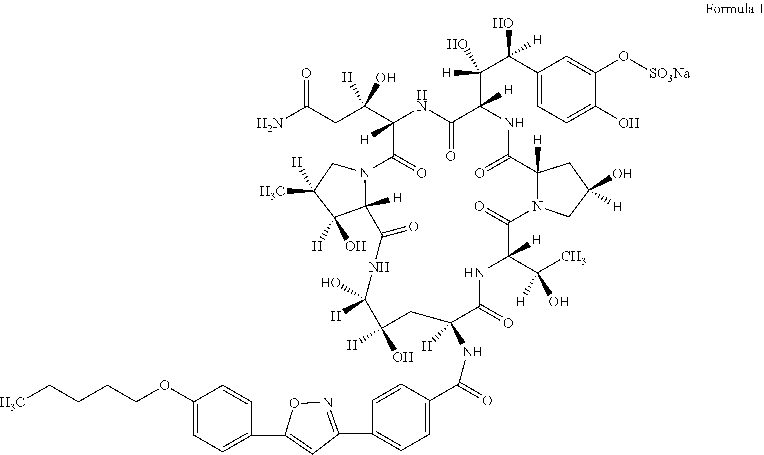

An objective of the present disclosure is to provide an echinocandin drug impurity and preparation and purification methods and use thereof.

In the preparation method of the present disclosure, adopted raw and auxiliary materials are easily available commercial materials, commercial micafungin is used as a starting material, and it is unexpectedly discovered that PH is adjusted to be in an acidic environment, and an HPLC purity of the echinocandin drug impurity in a reaction liquid is remarkably increased. The present disclosure simultaneously provides a method for further purification, during the purification, purification is performed by sequentially adopting a macroporous adsorptive resin and a silicagel column, so that the HPLC purity of the echinocandin drug impurity in the reaction liquid is further increased and reaches up to 90% or more, and a new concept is provided for industrial production. A requirement that a purity of a drug impurity reference substance needs to reach 90% or more is well known to the field. In the present disclosure, the echinocandin drug impurity with the purity being 90% or more is prepared for the first time, which may be used as a reference substance for drug quality control.

The echinocandin drug impurity provided by the present disclosure has a chemical name: 5-[(1S,2S)-2-[(3S,6S,9S,11R,15S,18S,20R,21S,24S,25S,26S)-3-[(R)-2-carbamoyl-1-ethoxyl]-11,20,21,25-tetr acarbonyl-15-[(R)-1-ethoxyl]-26-methyl-2,5,8,14,17,23-hexaoxo-18-[4-[5-(4-pentyloxy phenyl)isoxazole-3-yl]benzamido]-1,4,7,13,16,22-hexaazatricyclo[22.3.0.09,13]hepta-6-yl]-1,2-dihydroxyeth yl]-2-sodium hydroxyphenylsulfate, and see Formula I for its structure.

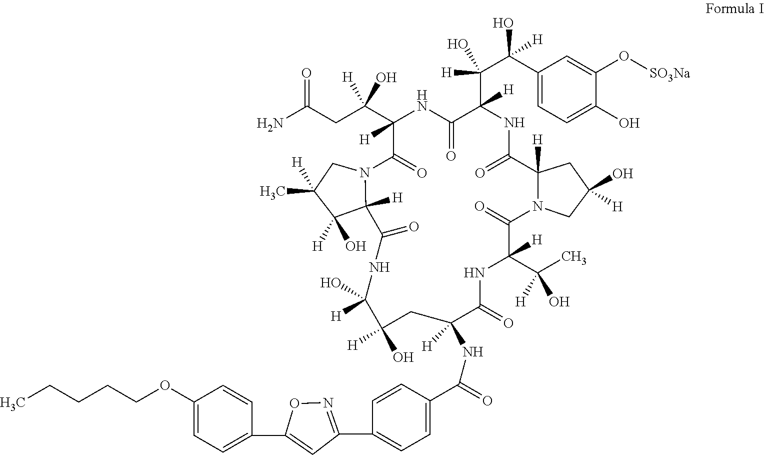

The preparation method of the present disclosure includes the following steps:
(a) preparing a protonic acid aqueous solution and adjusting a PH value of the aqueous solution; and
(b) adding the micafungin sodium into the protonic acid aqueous solution to be heated.

As a further improvement on the above preparation method, a protonic acid in step (a) is a molecule or an ion capable of giving protons (H′).

As a further improvement on the above preparation method, the protonic acid aqueous solution in step (a) is selected from the group consisting of a formic acid, a sulfuric acid, a hydrochloric acid, a phosphoric acid, an acetic acid, a sodium dihydrogen phosphate and any combination thereof, and pH of the solution is adjusted to be 1.0-6.0.

As a further improvement on the above preparation method, the protonic acid aqueous solution in step (a) is selected as an acetic acid; and pH of the solution is adjusted to be 2.0-3.0.

As a further improvement on the above preparation method, a reaction temperature in step (b) is 10-80° C.

As a further improvement on the above preparation method, a reaction temperature in step (b) is 20-50° C.

As a further improvement on the above preparation method, reaction time in step (b) is 148 h.

As a further improvement on the above preparation method, reaction time in step (b) is 10-24 h.

According to a preferred method for preparing the echinocandin drug impurity provided by the present disclosure, an HPLC purity in a reaction product is greater than 20%.

The present disclosure further provides a method for further purification, including producing a high-purity echinocandin drug impurity through column chromatography purification.

As a further improvement on the above preparation method, the adopted column chromatography purification includes macroporous adsorptive resin purification and silica gel purification.

The macroporous adsorptive resin purification specifically includes the following steps:
(a) performing adsorption on a reaction product through a macroporous adsorptive resin;
(b) performing elution by using an organic solvent to obtain a collected liquid; and
(c) performing elution again by using an organic solvent to obtain a collected liquid, and performing low temperature concentration.

As a further improvement on the above purification method, the adopted macroporous adsorptive resin in step (a) comprises styrene-divinylbenzene as a skeleton.

As a further improvement on the above purification method, the adopted macroporous adsorptive resin in step (a) includes HP20, HP20SS, HP21, SP70, SP700, SP825L, SP850, CHP20, CHP55 and any combination thereof, and the HP20SS macroporous adsorptive resin is preferred in the present disclosure.

As a further improvement on the above purification method, the organic solvent in step (b) is selected from the group consisting of methyl alcohol, ethyl alcohol, propyl alcohol, butanol, acetone, acetonitrile and any mixture thereof.

As a further improvement on the above purification method, in step (b), a volume percent of the organic solvent is 0% to 50% of a total volume of an eluant.

As a further improvement on the above purification method, in step (b), a volume percent of the organic solvent is 0% to 40% of a total volume of an eluant.

As a further improvement on the above purification method, in step (c), a volume percent of the organic solvent is preferred to be 70% to 100%.

As a further improvement on the above purification method, in step (c), a volume percent of the organic solvent is preferred to be 70% to 95%.

As a further improvement on the above purification method, a temperature is controlled to be 10-30° C. during the purification.

As a further improvement on the above purification method, a temperature is controlled to be 15-25° C. during the purification.

As a further improvement on the above purification method, a flow rate is 0.1-10 bed volumes per hour.

As a further improvement on the above purification method, a flow rate is 0.5-2 bed volumes per hour.

As a further improvement on the above purification method, in step (c), a temperature of the low temperature concentration is 5-25° C. and a concentrated solution containing echinocandin drug impurity components is obtained.

The silica gel purification specifically includes the following steps:
(a) preparing a loading buffer after diluting the above concentrated solution, and performing absorption through a silicagel column, and
(b) performing elution through an acidic aqueous solution of an organic solvent.

As a further improvement on the above purification method, the silica gel is a silica gel having a hydrophilic interaction chromatography mode (HILIC).

As a further improvement on the above purification method, in step (a), a proportion of the organic solvent in the loading buffer does not exceed 5%.

As a further improvement on the above purification method, the silica gel is selected from the group consisting of UniSil®, Chromatorex ARG Silica, Click XIon, Inertsil and any mixture thereof.

As a further improvement on the above purification method, the organic solvent is selected from the group consisting of methyl alcohol, ethyl alcohol, acetone, acetonitrile and any mixture thereof.

As a further improvement on the above purification method, a volume percent of the adopted organic solvent to water is 20% to 100%.

As a further improvement on the above purification method, a volume percent of the adopted organic solvent to water is 50% to 100%.

As a further improvement on the above purification method, the acidic solution is an aqueous solution selected from the group consisting of formic acid, an acetic acid, a hydrochloric acid, a phosphoric acid, a trifluoroacetic acid or a mixture thereof.

As a further improvement on the above purification method, a pH value of the acidic solution is 2.0-7.0.

As a further improvement on the above purification method, a pH value of the acidic solution is 3.0-5.0.

As a further improvement on the above purification method, the HPLC purity of the echinocandin drug impurity obtained through the chromatographic column purification is 90% or more.

Components of the collected liquid are concentrated in below 20° C., frozen and dried to obtain a finished echinocandin drug impurity.

A high-purity echinocandin drug impurity of a structure represented by Formula I may be applied to echinocandin drug quality control and is used as a reference substance for establishing an analysis method.

The high-purity echinocandin drug impurity refers to 90% or more of the HPLC purity.

### BRIEF DESCRIPTION OF THE DRAWINGS

FIG. 1 is a graph of hydrogen-nuclear magnetic resonance (¹H NMR) in Embodiment 5.

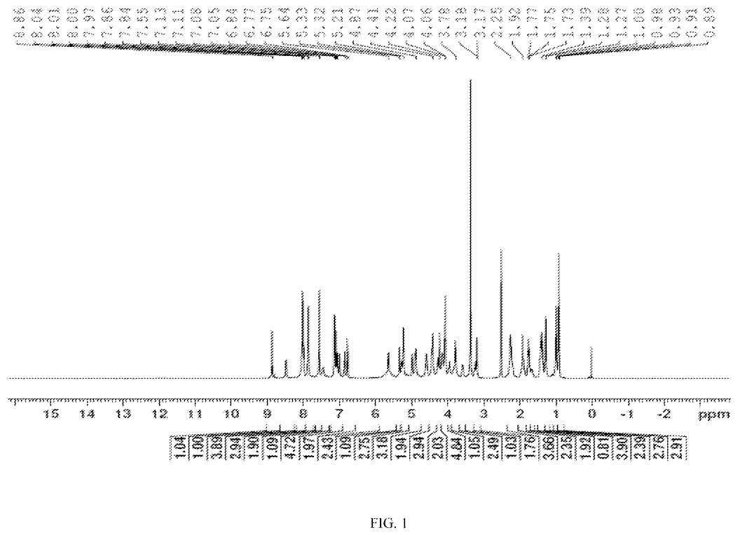

FIG. 2 is a graph of carbon-nuclear magnetic resonance (¹³C NMR) of an echinocandin drug impurity in Embodiment 5.

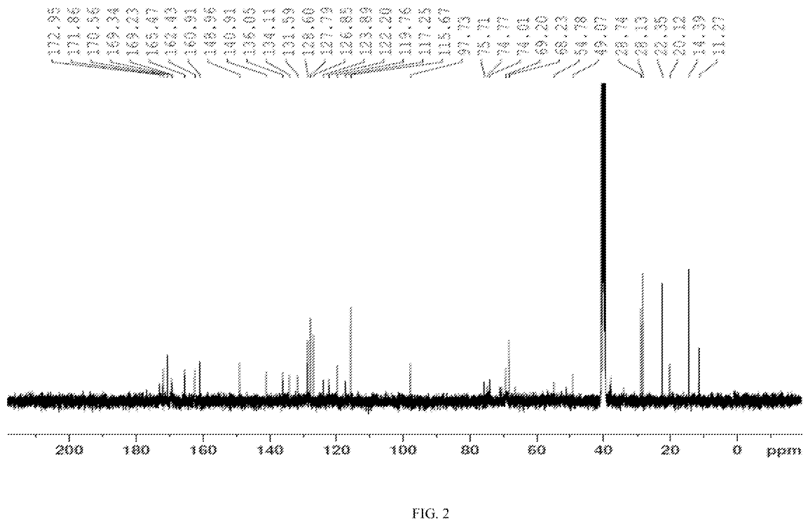

FIG. 3 is high resolution mass spectrometer (HRMS) of an echinocandin drug impurity in Embodiment 5.

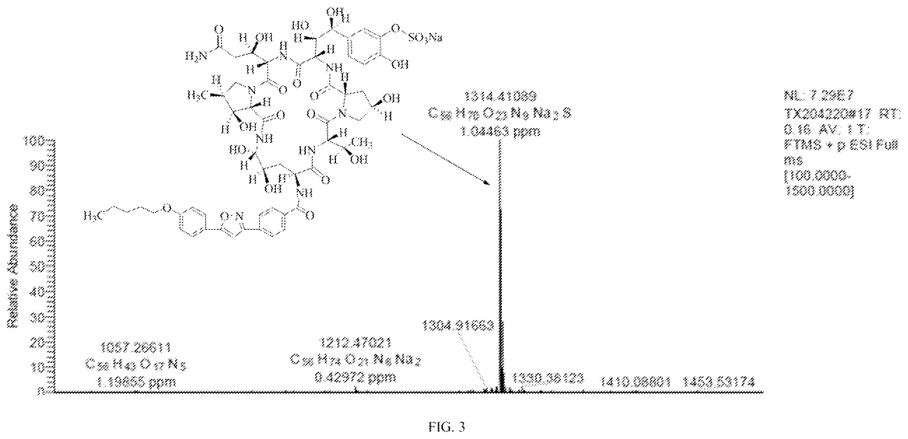

FIG. 4 is a graph of HPLC of an echinocandin drug impurity in Embodiment 5.

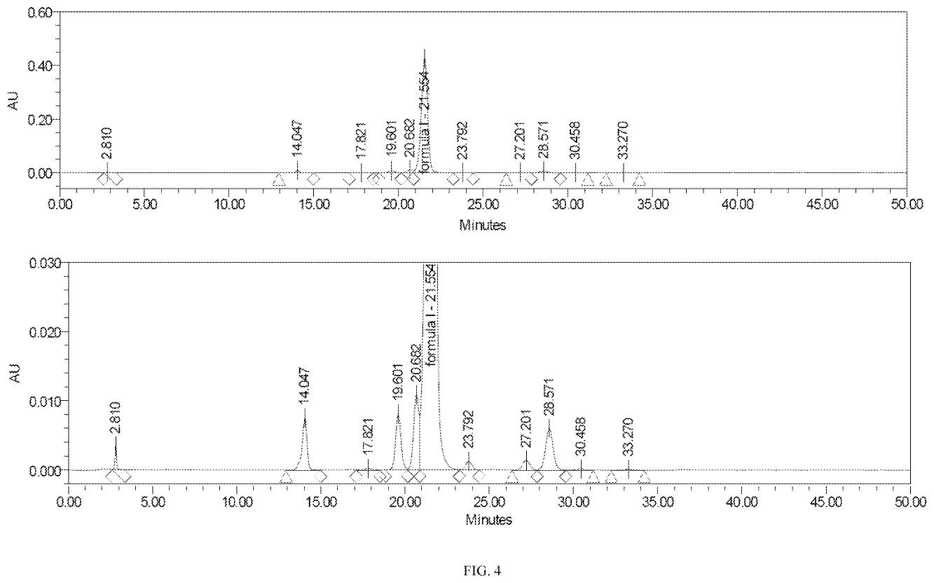

### DETAILED DESCRIPTION

The present disclosure is further described below with reference to specific embodiments.

### Comparison Example 1

Referring to literature [Journal of Synthetic Organic Chemistry 2006, 64, 12] and a patent [U.S. Pat. No. 6,107,458. U.S. Pat. No. 7,199,248], a micafungin sodium side chain (100 g) is dissolved in DMF (2.0 L), a temperature is decreased to 0° C., DIPEA (22.3 ml) is added, stirring is performed for 5 min, then FR-179642 (100 g) is added, and a thermal reaction is performed for 1.5 h. 1.0 L of methyl alcohol and acetone (V:V=1: 2) is added, then 12 L of ethyl acetate is added, stirring is performed for 12 h, then filtering is performed, and a micafungin compound is obtained. Purification is performed through a UBK resin, and a drug impurity containing 0.2% echinocandin is obtained. Through HP20SS column chromatography, a collected liquid is crystallized, then micafungin sodium is obtained, and a purity of the echinocandin drug impurity in the HPLC graph is 0.1%.

### Embodiment 1

100 mL of water is added into a reaction flask, and a concentrated hydrochloric acid is added dropwise slowly till PH reaches 1.0; and 1.0 g of micafungin sodium is added and stirred till solids are dissolved, a temperature of a reaction liquid is controlled to be 45° C., stirring is performed for 24 hours, sampling analysis is performed, and an HPLC purity of an echinocandin drug impurity in the reaction liquid is 3%.

### Embodiment 2

100 mL of water is added into a reaction flask, and a concentrated sulfuric acid is added dropwise slowly till PH reaches 2.0; and 1.0 g of micafungin sodium is added and stirred till solids are dissolved, a temperature of a reaction liquid is controlled to be 50° C., stirring is performed for 24 hours, sampling analysis is performed, and an HPLC purity of an echinocandin drug impurity in the reaction liquid is 8%.

### Embodiment 3

100 mL of water is added into a reaction flask, and a phosphoric acid is added dropwise slowly till PH reaches 4.0; and 1.0 g of micafungin sodium is added and stirred till solids are dissolved, a temperature of a reaction liquid is controlled to be 55° C., stirring is performed for 24 hours, sampling analysis is performed, and an HPLC purity of an echinocandin drug impurity in the reaction liquid is 12%.

### Embodiment 4

100 mL of water is added into a reaction flask, and an acetic acid is added dropwise slowly till PH reaches 3.0; and 1.0 g of micafungin sodium is added and stirred till solids are dissolved, a temperature of a reaction liquid is controlled to be 45° C., stirring is performed for 36 hours, and an HPLC purity of an echinocandin drug impurity in the reaction liquid is 23%.

### Embodiment 5

100 mL of water is added into a reaction flask, and an acetic acid is added dropwise slowly till PH reaches 2.8; and 1.0 g of micafungin sodium is added and stirred till solids are dissolved, a temperature of a reaction liquid is controlled to be 50° C., stirring is performed for 24 hours, and an HPLC purity of an echinocandin drug impurity in the reaction liquid is 21%. After the reaction is finished, absorption is performed by using 20 ml of HP20SS resin, washing is performed with 40 ml of 50% methyl alcohol, and elution is performed with 60 ml of 90% methyl alcohol. Collection, reduced pressure distillation and concentration are performed to obtain a collected liquid containing echinocandin drug impurity components. Then chromatographic separation is performed by using Click Xion silica gel, elution is performed by using 60% acetonitrile aqueous solution with PH being about 4.0, an echinocandin drug impurity is obtained, an HPLC purity is 92%, and 30 mg of white solids are obtained after freeze-drying is finished.

¹H NMR (DMSO-_{d6}, 400 MHz) δ: 0.91(t, J=8.0 Hz, 3H), 0.99 (d, J=8.0 Hz, 3H), 1.27 (d, J=4.0 Hz, 3H), 1.29-1.45 (m, 4H), 1.73-1.77 (m, 3H), 1.87-1.93 (m, 2H), 2.20-2.30 (m, 4H), 3.22 (t, J=4.0 Hz, 1H), 3.58-3.60 (m, 1H), 3.78 (s, 2H), 3.94 (m, 1H), 4.06-4.12 (m, 5H), 4.22 (m, 2H), 4.36-4.41 (m, 3H), 4.59 (m, 2H), 4.86-4.88 (m, 2H), 4.98 (s, 1H), 5.21-5.30 (m, 3H), 5.33 (d, J=4.0 Hz, 1H), 5.64 (d, J=4.0 Hz, 1H), 6.76 (d, J=8.0 Hz, 1H), 6.85 (d, J=8.0 Hz, 111), 6.99 (s, 1H), 7.05 (s, 1H), 7.08-7.20 (m, 4H), 7.45 (b, 1H), 7.55 (b, 2H), 7.84 (d, J=8.0 Hz, 2H), 8.00-8.04 (m, 4H), 8.46 (d, J=8.0 Hz, 1H), 8.86 (s, 1H).

¹³C NMR (DMSO-_{d6} 400 MHz) δ: 11.30, 14.39, 19.74, 22.35, 28.13, 28.74, 33.37, 37.62, 37.62, 38.61, 49.07, 51.60, 54.78, 56.80, 57.11, 57.66, 60.54, 66.30, 68.23, 69.20, 69.20, 69.79, 72.92, 73.86, 74.01, 74.77, 75.71, 97.73, 115.67, 115.67, 117.25, 119.76, 122.20, 123.87, 126.85, 126.85, 127.79, 127.79, 128.60, 128.60, 131.59, 134.11, 136.05, 140.91, 148.96, 160.91, 162.43, 165.47, 169.23, 169.34, 170.56, 170.56, 171.70, 171.90, 171.90, 172.95.

HRMS(ESI): calcd. For C₅₆H₇₀O₂₃N₉Na₂S(M+Na⁺): 1314.41089, Found: 1314.4095.

Table 1 Portion of composition result in an HPLC graph of echinocandin drug impurity in Embodiment 5

| | RT | Area | % Area | Height | JP Plate Count | Resolution |
|---|---|---|---|---|---|---|
| 1 | 2.807 | 23637 | 0.21 | 3422 | 4322 | 1.29 |
| 2 | 14.050 | 147805 | 1.31 | 7098 | 12987 | 13.85 |
| 3 | 16.778 | 3161 | 0.03 | 128 | 10961 | 3.36 |
| 4 | 17.822 | 7282 | 0.06 | 202 | 8041 | 1.46 |
| 5 | 19.605 | 173433 | 1.54 | 7600 | 17998 | 2.59 |
| 6 | 20.686 | 222730 | 1.97 | 10142 | | |
| 7 | 21.556 | 10435869 | 92.41 | 396467 | 16155 | |
| 8 | 23.796 | 31756 | 0.28 | 1154 | 18475 | 3.26 |
| 9 | 25.278 | 4099 | 0.04 | 110 | | |
| 10 | 27.208 | 41118 | 0.36 | 1283 | 16544 | |
| 11 | 28.576 | 174938 | 1.55 | 5591 | 19573 | 1.65 |
| 12 | 30.451 | 6373 | 0.06 | 171 | 19247 | 2.22 |
| 13 | 33.281 | 6345 | 0.06 | 165 | 17359 | 3.01 |
| 14 | 36.633 | 3724 | 0.03 | 95 | 19979 | 3.28 |

### Embodiment 6

100 mL of water is sequentially added into a reaction flask, and a formic acid is added dropwise slowly till PH reaches 3.0; and 1.0 g of micafungin sodium is added and stirred till solids are dissolved, a temperature of a reaction liquid is controlled to be 45° C., stirring is performed for 48 hours, and an HPLC purity of an echinocandin drug impurity in the reaction liquid is 20%. After the reaction is finished, absorption is performed by using 20 ml of HP20SS resin, washing is performed with 40 ml of 30% methyl alcohol, and elution is performed with 60 ml of 85% methyl alcohol. Collection, reduced pressure distillation and concentration are performed to obtain a collected liquid containing echinocandin drug impurity components. Then chromatographic separation is performed by using Chromatorex ARG Silica gel, elution is performed by using 70% acetonitrile aqueous solution with PH being about 6.0, an echinocandin drug impurity is obtained, an HPLC purity is 90%, and 60 mg of white solids are obtained after freeze-drying is finished.

### Embodiment 7

100 mL of water is sequentially added into a reaction flask, and an acetic acid is added dropwise slowly till PH reaches 3.0; and 1.0 g of micafungin sodium is added and stirred till solids are dissolved, a temperature of a reaction liquid is controlled to be 45° C., stirring is performed for 24 hours, and an HPLC purity of an echinocandin drug impurity in the reaction liquid is 8%. After the reaction is finished, absorption is performed by using 20 ml of SP207 resin, washing is performed with 40 mil of 1% ethyl alcohol, and elution is performed with 60 ml of 95% propyl alcohol. Collection, reduced pressure distillation and concentration are performed to obtain a collected liquid containing echinocandin drug impurity components. Then chromatographic separation is performed by using Inertsil silica gel, elution is performed by using 60% acetonitrile aqueous solution with PH being about 3.0, an echinocandin drug impurity is obtained, an HPLC purity is 91%, and 30 mg of white solids are obtained after freeze-drying is finished.

### Embodiment 8

100 mL of water is sequentially added into a reaction flask, and a phosphoric acid is added dropwise slowly till PH reaches 4.0; and 1.0 g of micafungin sodium is added and stirred till solids are dissolved, a temperature of a reaction liquid is controlled to be 80° C., stirring is performed for 48 hours, and an HPLC purity of an echinocandin drug impurity in the reaction liquid is 12%. After the reaction is finished, absorption is performed by using 20 ml of HP20SS resin, washing is performed with 40 ml of 50% methyl alcohol, and elution is performed with 70 ml of 95% ethyl alcohol. Collection, reduced pressure distillation and concentration are performed to obtain a collected liquid containing echinocandin drug impurity components. Then chromatographic separation is performed by using UniSil® silica gel, elution is performed by using 60% acetonitrile aqueous solution with PH being about 3.0, an echinocandin drug impurity is obtained, an HPLC purity is 91%, and 32 mg of white solids are obtained after freeze-drying is finished.

1. A high-purity echinocandin drug impurity of a structure represented by Formula I, wherein an HPLC purity of the impurity reaches 90% or more;

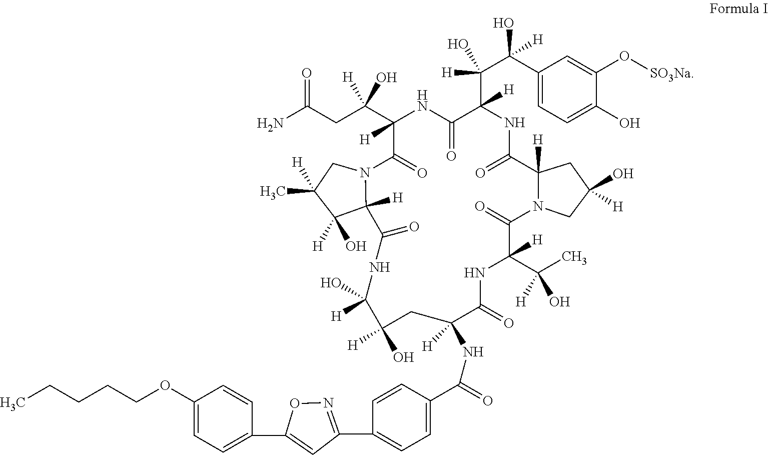

2. A method for preparing an echinocandin drug impurity of a structure represented by Formula I, comprising reacting micafungin sodium with a protonic acid aqueous solution to obtain a compound represented by Formula I.

3. The method of claim 2, wherein, the protonic acid is a molecule or an ion capable of giving protons, and the protonic acid is added to adjust pH of a reaction liquid to be acidic.

4. The method of claim 2, wherein, the protonic acid aqueous solution is selected from the group consisting of a formic acid, a sulfuric acid, a hydrochloric acid, a phosphoric acid, an acetic acid, a sodium dihydrogen phosphate and any combination thereof; and pH of a reaction liquid is adjusted to be 1.0-6.0.

5. The method of claim 2, wherein, the protonic acid aqueous solution is an acetic acid; pH of a reaction liquid is adjusted to be 2.0-3.0; and the compound represented by Formula I as a reaction product has an HPLC purity of greater than 20%.

6. A method for purifying an echinocandin drug Impurity of a structure represented by Formula I, comprising producing a high-purity echinocandin drug impurity through column chromatography purification.

7. The method of claim 6, wherein, the column chromatography purification comprises macroporous adsorptive resin purification and silica gel purification.

8. The method of claim 7, wherein, the macroporous adsorptive resin purification comprises steps of: a) performing adsorption on a reaction product through a macroporous adsorptive resin; b) performing elution by using an organic solvent to obtain a collected liquid; and c) performing elution again by using an organic solvent to obtain a collected liquid.

9. The method of claim 8, wherein, the macroporous adsorptive resin comprises styrene-divinylbenzene as a basic skeleton.

10. The method of claim 9, wherein, the macroporous adsorptive resin is selected from the group consisting of HP20, HP20SS, HP21, SP70, SP700, SP825L, SP850, CHP20, CHP55 and any combination thereof.

11. The method of claim 8, wherein, the organic solvent is selected from the group consisting of methyl alcohol, ethyl alcohol, propyl alcohol, butanol, acetone, acetonitrile and any mixture thereof.

12. The method of claim 8, wherein, in step b), a volume percent of the organic solvent is 0% to 50% of a total volume of an eluant, preferably 0% to 40%.

13. (canceled)

14. The method of claim 8, wherein, in step c), a volume percent of the organic solvent is 70% to 100%, preferably 70% to 95%.

15. (canceled)

16. The method of claim 8, wherein, a temperature is controlled to be 10-30° C. during the purification, preferably 15-25° C.

17. (canceled)

18. The method of claim 7, wherein, the silica gel purification comprises steps of: performing absorption on a collected liquid containing drug impurity components obtained after the macroporous adsorptive resin purification through a silica gel column, and performing elution through an acidic aqueous solution of an organic solvent.

19. The method of claim 18, wherein, the silica gel is a silica gel having a hydrophilic interaction chromatography mode.

20. The method of claim 18, wherein, the silica gel is selected from the group consisting of UniSil®, Chromatorex ARG Silica, Click XIon, Inertsil and any combination thereof.

21. The method of claim 18, wherein, the organic solvent is selected from the group consisting of methyl alcohol, ethyl alcohol, acetone, acetonitrile and any mixture thereof.

22. The method of claim 21, wherein, a volume percent of the organic solvent is 20% to 100%, preferably 50% to 100%.

23. (canceled)

24. The method of claim 18, wherein, the acidic solution is an aqueous solution of a formic acid, an acetic acid, a hydrochloric acid, a phosphoric acid, a trifluoroacetic acid or a mixture thereof.

25. The method of claim 24, wherein, a pH value of the acidic solution is 2.0 to 7.0, preferably 3.0-5.0.

26. (canceled)

27. The method of claim 7, comprising: performing absorption by using an HP20SS resin, performing washing with 50% methyl alcohol, performing elution with 90% methyl alcohol, and performing collection, reduced pressure distillation and concentration to obtain a collected liquid containing echinocandin drug impurity components; and performing chromatographic separation by using a Click Xion silica gel, and performing elution by using 60% acetonitrile aqueous solution of PH 4.0 to obtain an echinocandin drug impurity.

28. A method for echinocandin drug quality control, comprising using a high-purity echinocandin drug impurity of a structure represented by Formula I as a reference substance for establishing an analysis method.
